(19) United States
(12) Patent Application Publication
(10) Pub. No.: US 2022/0096045 A1
(43) Pub. Date: Mar. 31, 2022

(54) BREAST SCREENING EQUIPMENT

(71) Applicant: HITACHI, LTD., Tokyo (JP)

(72) Inventors: Fuka MATSUDAIRA, Tokyo (JP); Ai MASUDA, Tokyo (JP); Narumi NAGAHAMA, Tokyo (JP); Yayoi KONISHI, Tokyo (JP); Takahide TERADA, Tokyo (JP); Yushi TSUBOTA, Tokyo (JP)

(21) Appl. No.: 17/227,416

(22) Filed: Apr. 12, 2021

(30) Foreign Application Priority Data
Sep. 28, 2020 (JP) 2020-162646

(51) Int. Cl. A61B 8/08; A61B 8/00

(52) U.S. Cl. CPC A61B 8/0825; A61B 8/44; A61B 8/461

(57) ABSTRACT

A breast screening equipment includes: a support table to support a subject in a prone position; and an examination mechanism having a cylinder which has an opening at an upper end so that a breast to be examiner is inserted and be protruded from an upper surface of the support table, wherein the examination mechanism conducts an ultrasound examination of the inserted breast in the cylinder. The cylinder includes a hollow portion which is located inside of a wall constituting the cylinder and is extended above the upper surface of the support table. The examination mechanism includes a ring-shaped ultrasound transmission reception mechanism being movable inside of the hollow portion vertically.

### RELATED APPLICATION

The present application is based on, and claims priority from, Japanese application No. 2020-162646, filed Sep. 28, 2020, the disclosure of which is hereby incorporated by reference in its entirety.

### BACKGROUND

### Technical Field

This disclosure relates to a breast screening equipment.

### Related Art

Breast screening such as a breast cancer test is often quite painful to a subject. Given the situation, there has been a demand for development of a new breast screening equipment. International Publication WO 2019/069898 (claim 1) (Patent Document 1) discloses a medical imaging apparatus including: a bath tank for accommodating a test subject, which is at least a portion of a body of a human subject; a measurement device movable in a predetermined direction, the measurement device including a group of elements to emit a radiation wave into the bath tank and receive a scattered radiation wave; and a control unit for measuring by the measurement device when at least one of a location of the test subject within a plane orthogonal to the predetermined direction, a location of the test subject in the predetermined direction, and continuity of data measured by the measurement device satisfies a predetermined condition.

### SUMMARY

According to the medical imaging apparatus disclosed in Patent Document 1, a breast of a human subject in a prone position is inserted into an opening, and the breast undergoes ultrasound examination inside the opening (FIG. 3).

However, there may be a human subject who cannot insert the breast fully into the opening, and a measurement range may be restricted in this case. This disclosure is directed to a breast screening equipment which is capable to examine a wide range.

A breast screening equipment according to an aspect of this disclosure includes: a support table configured to support a subject in a prone position; and an examination mechanism including a cylinder having an opening at an upper end and protruding from an upper surface of the support table, wherein a breast to be examine of the subject is inserted in the opening and the examination mechanism is configured to conduct an ultrasound examination of the inserted breast inside the cylinder.

According to this disclosure, it is possible to provide a breast screening equipment which is capable to examine a wide range.

### BRIEF DESCRIPTION OF THE DRAWINGS

FIG. 1 is a perspective view of a breast screening equipment of an embodiment.

FIG. 2 is a diagram for explaining an examination method by using an examination mechanism, which is a cross-sectional view in a front-rear direction including a cylinder of the examination mechanism.

FIG. 3 is a top plan view of the examination mechanism.

FIG. 4 is a cross-sectional view taken along the A-A line in FIG. 1.

FIG. 5 is a top plan view of the breast screening equipment of the embodiment.

FIG. 6 is a side view of the breast screening equipment of the embodiment.

FIG. 7 is a block diagram of the breast screening equipment of the embodiment.

FIG. 8 is a schematic diagram of a first screen.

FIG. 9 is a schematic diagram of a second screen.

FIG. 10 is a schematic diagram of a third screen.

FIG. 11 is a flowchart showing a breast screening method of the embodiment.

### DETAILED DESCRIPTION OF THE EMBODIMENTS

Modes for carrying out this disclosure (hereinafter referred to as embodiments) will be described below with reference to the drawings. In the following description of one embodiment, other embodiments applicable to the one embodiment will also be described therein as appropriate. This disclosure is not limited only to the one embodiment, but may be realized by combining different embodiments with each other or modified as desired within a range not significantly damaging effects of this disclosure. In the meantime, the same constituents will be denoted by the same reference signs and overlapping explanations thereof will be omitted. Moreover, the constituents having the same function will basically have the same name. The illustrated contents are merely schematic and may be changed from actual configurations for the convenience of illustration to the extent not significantly damaging the effects of this disclosure.

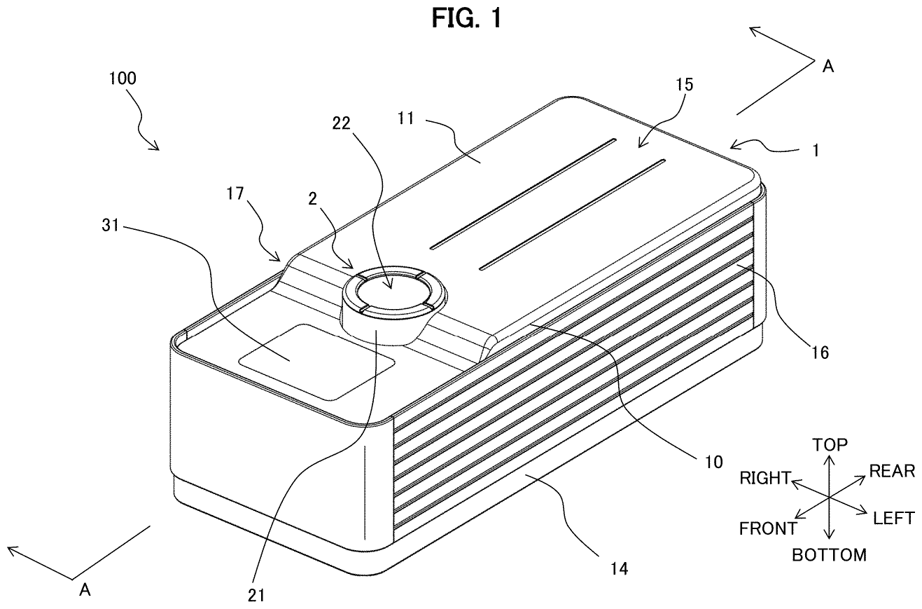

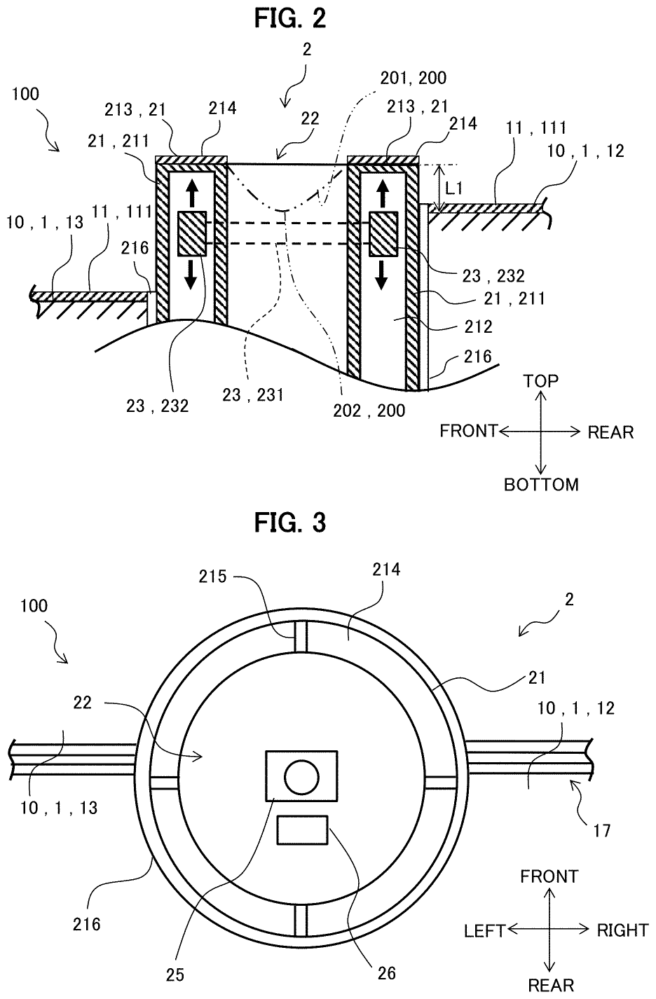

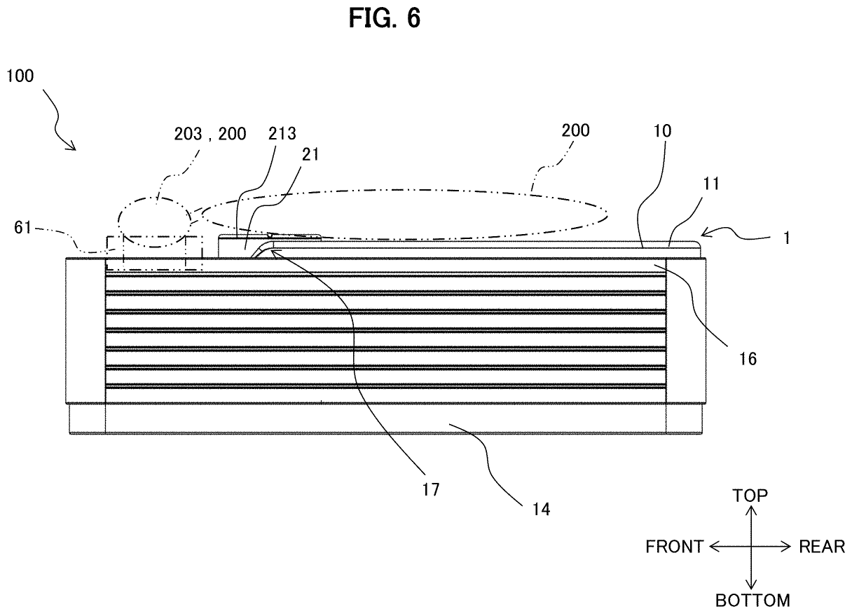

FIG. 1 is a perspective view of a breast screening equipment 100 of this embodiment. The breast screening equipment 100 discover a disease such as a breast cancer by conducting ultrasound examination of a breast 201 (FIG. 2) inside a cylinder 21. The breast screening equipment 100 has a substantially rectangular parallelepiped shape in such a size that enables a subject 200 (FIG. 6) to lie thereon in a state of a prone position.

The breast screening equipment 100 includes a support table 1 that supports the subject 200 in the prone position and an examination mechanism 2 to perform ultrasound examination of the breast 201. The support table 1 is constructed at such a height that allows the subject 200 to sit thereon and to lie down thereon in a front-rear direction. The height of the support table 1 varies in the front-rear direction, and a front side part of the support table 1 is connected to a rear side part thereof through a slope 17 that smoothly goes down from the rear side part to the front side part. The slope 17 extends in a right-left direction.

A cushion 11 formed from an elastic body such as low repulsion urethane is arranged on an upper surface 10 (note that FIG. 1 illustrates only right and left end portions on a rear side of the upper surface 10; see FIG. 2) of the support table 1. The cushion 11 deforms in a yielding manner when the subject 200 lies down thereon, so that cushion 11 reduces pain to the body. The cushion 11 is arranged in the same thickness, for example, so as to cover the entire region of the upper surface 10 in the front-rear direction and the right-left direction.

In another embodiment, the examination mechanism 2 includes an adjustment mechanism (not shown) that adjusts a position in a vertical direction of the cylinder 21 (to be described later). The height of the cylinder 21 sensed by the subject 200 in the prone position is adjusted by causing the adjustment mechanism to adjust the position in the vertical direction of the cylinder 21. This makes it possible to conduct examination while reducing the pain that may be experienced by the subject 200.

In still another embodiment, the breast screening equipment 100 includes two or more cushions 11 having different thicknesses. The height of the cylinder 21 sensed by the subject 200 in the prone position is adjusted by changing the thicknesses of the cushion 11 to be placed. This makes it possible to conduct the examination while reducing the pain that may be experienced by the subject 200.

In yet another embodiment, the breast screening equipment 100 includes two or more cushions 11 having the same thickness. The height of the cylinder 21 sensed by the subject 200 in the prone position can be adjusted by changing the number of the cushions 11 to be placed. This makes it possible to conduct the examination while reducing the pain that may be experienced by the subject 200.

Back to FIG. 1, a positioning guide 15 serving as a mark for the subject 200 lying down is formed on an upper surface of the cushion 11. The positioning guide 15 is formed from, for example, grooves which extend in the front-rear direction. In the case of examining the right breast 201 (FIG. 2), for instance, the subject 200 (FIG. 6) easily inserts the right breast 201 into an opening 22 of the cylinder 21 by lying down on the support table 1 in such a way as to align the center of the body (the body axis) with the groove on the left side.

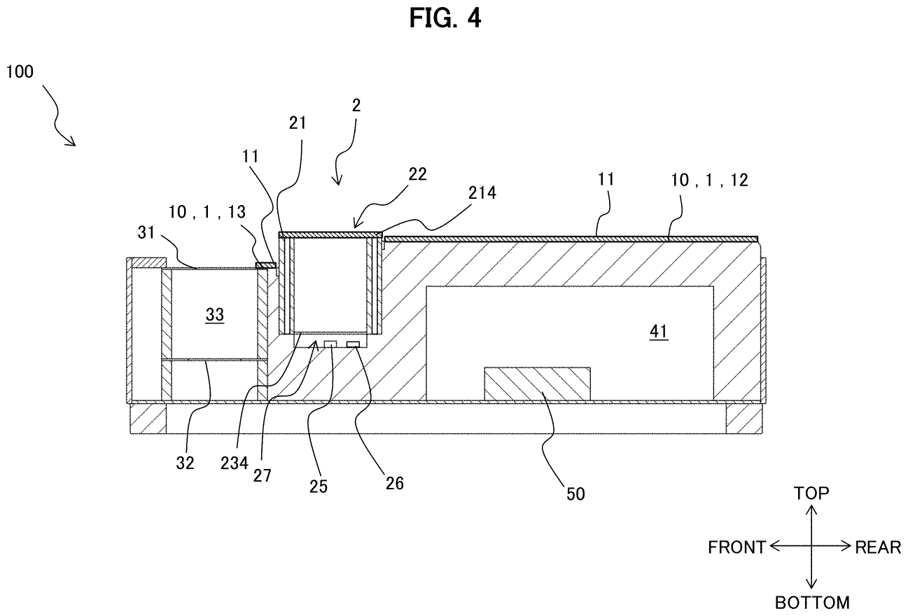

The breast screening equipment 100 includes side covers 16 on right and left side surfaces. A control device 50 (FIG. 4) and the like installed inside are accessible and maintainable by detaching any of the side covers 16. The breast screening equipment 100 includes a riser cover 14 located below the side covers 16 for covering legs (not shown) to support the breast screening equipment 100.

FIG. 2 is a diagram for explaining a screening method by using the examination mechanism 2, which is a cross-sectional view in a front-rear direction including the cylinder 21 of the examination mechanism 2. The examination mechanism 2 includes the cylinder 21, which has the opening 22 located at an upper end for allowing insertion of the breast 201 as an examination subject of the subject 200 (FIG. 6), and is arranged in a projecting manner from the upper surface 10 of the support table 1. In order to subject the inserted breast 201 to the ultrasound examination inside the cylinder 21, the cylinder 21 is formed from a rigid material (such as a metal and a resin) so that the cylinder 21 supports the subject 200. In FIG. 2, illustration of part of the body of the subject 200 is omitted and only the breast 201 and a nipple 202 are illustrated therein. By inserting the breast 201 into the cylinder 21 that projects from the upper surface 10, it is possible to bring the breast 201 into close contact with the cylinder 21 and to prevent a movement of the breast 201 during the examination.

The cylinder 21 is a circular cylinder which has either an exactly circular shape or an oval shape in top plan view. In the example illustrated in FIG. 2, the cylinder 21 has an exactly circular shape in top plan view. An annular cushion 214 corresponding to a shape of an upper end surface 213 of the cylinder 21 is placed on the upper end surface 213. The cushion 214 is formed from of the same material and into the same thickness as the cushion 11, for example. The use of the cushion 214 reduces the pain that the subject 200 may experience when she inserts the breast 201 into the opening 22.

The cylinder 21 includes a hollow portion 212, which is located inside a wall 211 that constitutes the cylinder 21, and extends above the upper surface 10 of the support table 1. In particular, the hollow portion 212 extends above a first surface 12 that supports a lower body side relative to the breasts 201 of the subject 200, and extends further above an upper surface 111 of the cushion 11 that is placed on the upper surface 10. The examination mechanism 2 includes an ultrasound transmission reception mechanism 23 in a ring shape that moves vertically inside the hollow portion 212. Provision of the ultrasound transmission reception mechanism 23 enables an examination of a portion of the breast 201 located above the upper surface 10 of the support table 1.

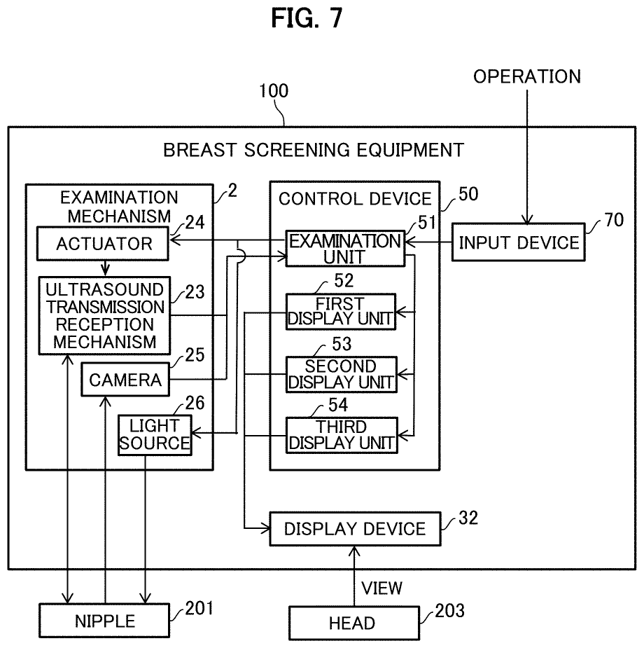

The ultrasound transmission reception mechanism 23 includes a support body 231 and transmission-reception probes 232. The ultrasound transmission reception mechanism 23 is formed by arranging the transmission-reception probes 232 for transmitting and receiving ultrasound at equal intervals on the support body 231 formed into an annular shape. By causing the transmission-reception probes 232 to transmit and receive the ultrasound while moving the support body 231 vertically by use of an actuator 24 (FIG. 7), it is possible to conduct ultrasound examination of cross-sections (cross-sections extending in the front-rear direction and the right-left direction) of the breast 201 inserted into the cylinder 21.

The examination mechanism 2 is configured to conduct the ultrasound examination in a state of putting an examination liquid into the cylinder 21. The examination liquid is capable of propagating ultrasound and is preferably colorless and transparent. The examination liquid is water such as cold water and warm water, or more preferably, lukewarm water at a temperature in a range from 30° C. to 35° C., for example. By conducting the ultrasound scan in the state of putting the examination liquid therein, it is possible to improve an SN ratio and thus to improve accuracy of examination.

In another embodiment, the examination mechanism 2 includes a liquid level detection mechanism (not shown) configured to detect a liquid level inside the cylinder 21. For example, after inserting the breast 201 into the opening 22, the examination liquid is poured by using an examination liquid pouring mechanism (not shown) until the liquid level detected by the liquid level detection mechanism reaches the opening 22. Alternatively, the breast 201 may be inserted after the pouring mechanism pours the examination liquid to a liquid level lower than the breast 201, and then the examination liquid may additionally be poured until the liquid level reaches the opening 22, for example.

The ultrasound transmission reception mechanism 23 located at the highest position may serve as the liquid level detection mechanism, for example. When the examination liquid is poured while transmitting and receiving the ultrasound with the ultrasound transmission reception mechanism 23, behaviors of the ultrasound vary at a liquid surface of the examination liquid. Thus, the liquid level is determined by detecting the variation in behaviors. Meanwhile, the examination mechanism 2 includes a camera 25 (FIG. 4) located on a bottom side of the cylinder 21 and configured to take an image of the breast 201. Details of the camera 25 will be described later. The examination mechanism 2 detects the liquid level by taking an image of an area above the camera 25 with the camera 25. The use of the camera 25 enables detection of the liquid level without having to use an independent sensor.

In another case, the liquid level detection mechanism is a liquid level sensor (not shown) provided to the opening 22, for instance. Alternatively, the liquid level detection mechanism may be a liquid detection sensor (not shown) provided on an outer side surface of the cylinder 21, for example. By detecting a leakage of the examination liquid with the liquid detection sensor, it is possible to stop pouring the examination liquid when the cylinder 21 is filled with the examination liquid.

Back to FIG. 2, a height L of the cylinder 21 relative to the upper surface 10 of the support table 1 is set to a predetermined height that hardly causes pain to the subject 200. In this way, it is possible to let the subject 200 feel less pain when she inserts the breast 201 into the opening 22. Here, the height L does not include the thickness of the cushions 11 and 214 because the cushions 11 and 214 are deformed in conformity with the subject 200 when she lies down and are barely involved in the pain to her.

In particular, the height L (a predetermined height) of the cylinder 21 relative to the upper surface 10 of the support table 1 is in a range from 5 mm or more to 50 mm or below. In this way, it is possible to reduce the pain to the subject 200 at the timing of putting her breast on the opening 22 in the case that she inserts her breast into the opening 22. In particular, when the subject 200 is in the prone position, the cushions 11 and 214 are deformed in conformity therewith as mentioned above. Accordingly, it is preferable to set the height of the cushion 214 relative to an upper surface of the cushion 11 in the aforementioned range in the state where the subject 200 is in the pone position.

The height L (which may be the height of the cushion 214 relative to the upper surface of the cushion 11) is in a range from 10 mm or more to 35 mm or below or preferably in a range from 15 mm or more to 25 mm or below. Particularly by setting the height L particularly in the range from 15 mm or more to 25 mm or below, even when the subject 200 has an ample figure or has large bust, it is possible to prevent the body from being lifted up by the fat around her abdominal area or by the breasts 201 while preventing the pain. As a consequence, the subject 200 inserts the breast 201 deep into the cylinder 21.

In another embodiment, the height L of the cylinder 21 relative to the upper surface 10 of the support table 1 is set to the height L obtained by acquiring heights L as data in advance at which multiple subjects 200 barely feel pain, and determining such a height L based on the data at which, for example, 90% or more of the subjects 200 barely feel pain, as the height L of the cylinder 21. It is possible to directly ask each subject 200 whether or not she feels pain. Alternatively, the degree of pain is determined based on a measurement value by using a pressure sensor (not shown) installed on the upper end surface 213 of the opening 22, for example.

Back to FIG. 2, the upper surface 10 includes the first surface 12 to support the lower body of the subject 200, and a second surface 13 to lay the head 203 (FIG. 6) of the subject 200, where the surfaces are arranged, respectively, on two sides of the cylinder 21 in lateral view. The second surface 13 is located at a lower position than the first surface 12. In the example shown in FIG. 2, the first surface 12 is located at a higher position than the upper surface 111 of the cushion 11 to be placed on the second surface 13.

In this way, it is possible to place the shoulders, the collarbones, and the heads of humeri of the subject 200 on the lowered second surface 13 side when the subject 200 lies down so as to insert the breast 201 into the opening 22. This makes it possible to prevent the shoulders, the collarbones, and the heads of humeri of the subject 200 from contacting, with the upper surface 10 so as to enable insertion of the breast 201 deep into the cylinder 21 while avoiding the lift of the body.

Meanwhile, the first surface 12 is smoothly connected to the second surface 13 by using the slope 17 (FIG. 1). The cylinder 21 is located at the same position as the slope 17 (see FIG. 1 as well). To be more precise, an upper end of the slope 17 is located near the center of the cylinder 21 that is formed into the exactly circular shape in top plan view (FIG. 3, for example).

FIG. 3 is a top plan view of the examination mechanism 2. The examination mechanism 2 includes flow channels 215 that flows the examination liquid overflowing from the opening 22 from the inside of the cylinder 21 to the outside of the cylinder 21. The flow channels 215 are, for example, grooves which are formed in an upper surface of the cushion 214 placed on the upper end surface 213 (FIG. 2) of the cylinder 21. Provision of the flow channels 215 makes it possible to flow the examination liquid overflowing from the cylinder 21 at the time of pouring the examination liquid into the cylinder 21 or during the examination, to the outside of the cylinder 21 through the flow channels 215.

The flow channels 215 are formed in such a way as to flow the examination liquid to positions around the armpits of the subject 200 when the subject 200 lies down. Specifically, in light of FIG. 3, for instance, the positions around the armpits of the subject 200 are defined by angles in a forward direction in a range from 30° or more to 150° or lower or preferably in a range from 60° or more to 120° or lower on the right and left, respectively. Since the portions around the armpits of the subject 200 are not in close contact with the support table 1, the flow channels 215 are less likely to be blocked so that the examination liquid is flowed easily.

The flow channels 215 are formed in a bilaterally symmetrical fashion. Accordingly to this configuration, the subject 200 lies over the left side of the flow channel 215 at the time of examining the right breast 201 and the flow channel 215 on the left side is blocked as a consequence. However, the examination liquid still flows through the flow channel 215 on the right side. The same applies to the case of examining the breast 201 on the left side.

In the example shown in FIG. 3, the four flow channels 215 are formed in total in such a way as to extend straight in radial directions of the circular cylinder 21 at regular intervals of 90° and in the bilaterally symmetrical fashion.

The examination mechanism 2 includes a receiving portion 216 provided in such a way as to surround at least a part of the cylinder 21 and designed to receive the examination liquid that flows out through the flow channels 215. The receiving portion 216 is formed in such a way as to overlap the positions of the flow channels 215 in top plan view. In the example illustrated in FIG. 3, the receiving portion 216 is formed annularly so as to surround the entire circumference of the cylinder 21. The receiving portion 216 is, for example, a groove (a trench) and a lower part of the receiving portion 216 is connected to a tank (not shown). Thus, the examination liquid that flows out from the flow channels 215 is stored in the tank. In the meantime, it is possible to allow the examination liquid to flow in the receiving portion 216 so as to prevent a leakage to the support table 1, thus reducing uncomfortable feeling of the subject 200 associated with the leakage. In addition, it is possible to prevent a window 31 (FIG. 4) from being wet due to the leakage so as to allow the subject 200 to view a display device 32 (FIG. 4).

In another embodiment, the annular receiving portion 216 has a slope that goes down forward, and the receiving portion 216 includes a discharge outlet (not shown), which is located at a lower surface at the lowest portion of the receiving portion 216 and is connected to the tank. The discharge outlet is covered with a mesh member. The examination liquid that flows out to the receiving portion 216 through the flow channels 215 flows along the slope and further flows into the tank through the discharge port at the lowest portion. By forming the receiving portion 216 as described above, it is possible to remove an extraneous material in case the material is dropped on the receiving portion 216.

In still another embodiment, a width (a length in the radial direction of the cylinder 21 formed into a circular tube) of the receiving portion 216 is set to such a width that facilitates insertion of human fingers. In this way, the receiving portion 216 is easily cleaned by putting a cloth into the receiving portion 216, for example.

In yet another embodiment, the receiving portion 216 does not have to be provided. In this case, the examination liquid that flows out through the flow channels 215 is wiped off with, for example, a towel so as to reduce uncomfortable feeling of the subject 200.

Back to FIG. 3, the breast screening equipment 100 includes the camera 25 and a light source 26 which are located below the opening 22. Positions of the camera 25 and the light source 26 will be described with reference to FIG. 4.

FIG. 4 is a cross-sectional view taken along the A-A line in FIG. 1. The cylinder 21 has a bottom and includes a bottom plate 234 made of, for example, a colorless transparent resin. The examination liquid poured into the cylinder 21 stays above the bottom plate 234. The breast screening equipment 100 includes a space 27 defined below the bottom plate 234. The camera 25 for shooting an image of the breast 201 (FIG. 2) through the bottom plate 234 and the examination liquid, and the light source 26 that illuminates the breast 201 during the image shooting are arranged in the space 27.

The camera 25 shoots the image of the breast 201 while taking into account a variable view angle because the view angle may change due to the bottom plate 234 and the examination liquid. The light source 26 illuminates the breast 201 with light that improves imaging sensitivity of the camera 25. The light source 26 emits visible light or infrared light, for example. When the camera 25 is capable of shooting an image in a bright place, the light source 26 emits the visible light, for example. Although details will be described later, after the breast 201 is inserted into the opening 22, positions of the breast 201 (FIG. 2) and the nipple 202 (FIG. 2) are adjusted before the ultrasound examination. In the position adjustment, a video image or a still image shot with the camera 25 is mirror reversed, and the display device 32 displays either the video image or the still image that is mirror reversed.

The breast screening equipment 100 is provided with the display device 32 visible to the subject 200, which is located at a position corresponding to the head 203 (FIG. 6) of the subject 200 (FIG. 6) in top plan view. By providing the display device 32, it is possible to transmit information in a visual way to the subject in the prone position. The display device 32 is a liquid crystal display unit, for example. The breast screening equipment 100 includes the window 31 made of a colorless transparent resin, for example, and a space 33 is defined at least between the window 31 and the display device 32. The air, for example, is present in the space 33. The subject views the display device 32 through the window 31 and the space 33. Accordingly, it is possible to secure a wide space from the head 203 to the display device 32 and thus to improve visibility.

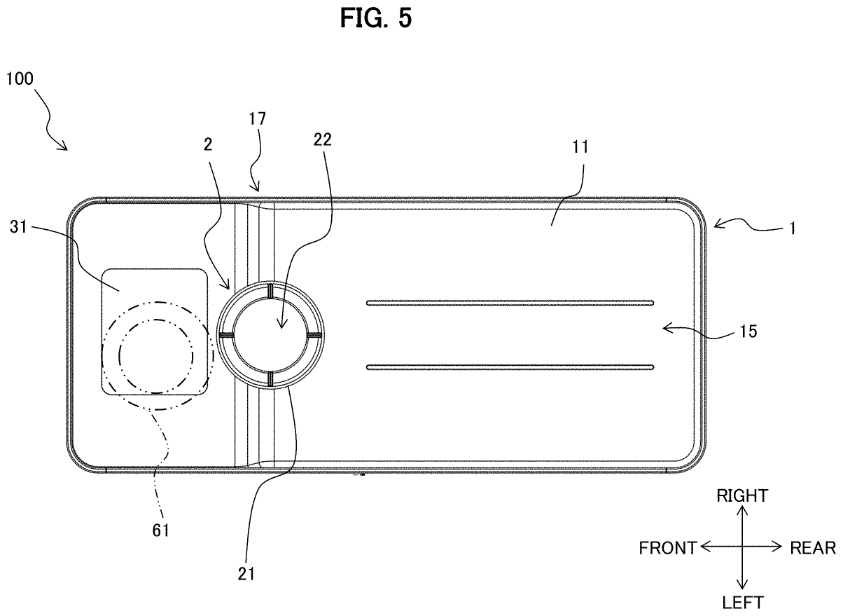

FIG. 5 is a top plan view of the breast screening equipment 100 of this embodiment. Meanwhile, FIG. 6 is a side view of the breast screening equipment 100 of this embodiment. During the examination, an annular face pillow 61 formed from a soft material such as a cushion is placed on the window 31. The face pillow 61 is movable in up-down directions and right-left directions as appropriate depending on the position of the head 203 (FIG. 6) of the subject 200. The face pillow 61 has substantially the same height as the height of the upper end surface 213 (FIG. 6) of the cylinder 21 when placed on the window 31. During the examination, the subject 200 takes a prone position along the positioning guide 15 corresponding to the breast 201 to be examined and places the head203 on the face pillow 61. Thus, the subject 200 views the display device 32 through the window 31.

Back to FIG. 4, the shape of each of the window 31 and the display device 32 is formed into a rectangular shape in which a length in the right-left direction is larger than a length in the up-down direction. The window 31 is formed larger than the size of the display device 32, so that the subject 200 views the display device 32 through the window 31 even when she inserts any of the right and left breasts 201 into the opening 22.

The breast screening equipment 100 includes the control device 50 located on the opposite side of the display device 32 viewed from the examination mechanism 2 and conducts a drive control of the breast screening equipment 100. The control device 50 is arranged in a space 41 defined below the first surface 12 and is connected to a power unit (not shown). The control device 50 will be described with reference to FIG. 7.

FIG. 7 is a block diagram of the breast screening equipment 100 of this embodiment. The control device 50 controls operation of the breast screening equipment 100. An input device 70 provided with buttons and a keyboard, for example, is connected to the control device 50. The control device 50 drives the breast screening equipment 100 in accordance with inputted contents inputted to the input device 70 by an operator (not shown, such as an ultrasonologist). For example, when the operator inputs a examination start instruction by pressing a button (not shown), the control device 50 performs pouring of the examination liquid into the cylinder 21 (FIG. 2), driving of the ultrasound transmission reception mechanism 23 in the vertical direction as well as ultrasound transmission and reception, display of predetermined screens on the display device 32, and the like in accordance with given programs stored in advance.

For example, the control device 50 includes a central processing unit (CPU), a random access memory (RAM), a read only memory (ROM), and the like through none of them are illustrated. The control device 50 is realized by loading a prescribed control program stored in the ROM into the RAM and causing the CPU to execute the program.

The control device 50 includes an examination unit 51 that performs the examination of the breast 201 by using the examination mechanism 2, a first display unit 52, a second display unit 53, and a third display unit 54. The examination unit 51 examines the breast 201 (FIG. 2) by controlling the examination mechanism 2 based on the input of the examination start instruction to the input device 70.

Based on the instruction from the examination unit 51, the first display unit 52 causes the display device 32 to display a first screen 521 that guides a position of at least one of the breast 201 (FIG. 2) and the nipple 202 (FIG. 2) of the subject 200 (FIG. 6) in the prone position. Provision of the first display unit 52 allows the subject 200 in the prone position to easily adjust the position of the breast 201 or the nipple 202 inside the cylinder 21.

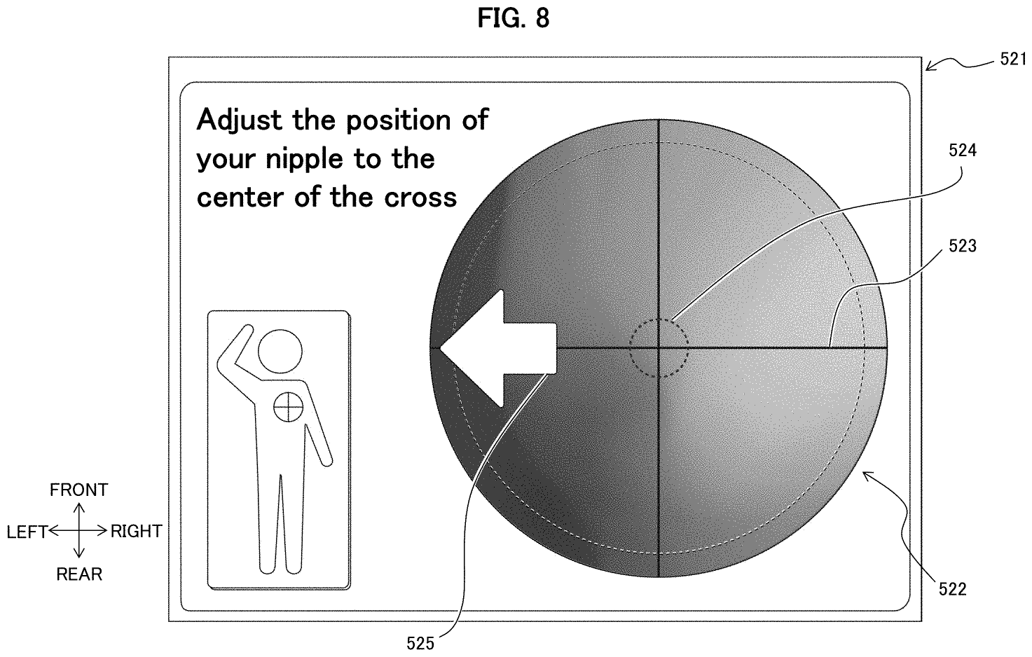

FIG. 8 is a schematic diagram of the first screen 521. The first screen 521 includes a guiding section 522 on the right side for guiding the positions of the breast 201 (FIG. 2) and the nipple 202 (FIG. 2). The guiding section 522 has a circular shape and includes reference lines 523 that extend in the up-down direction and the right-left direction, respectively, and a center circle 524 located at an intersection of the reference lines 523. The subject 200 moves her body while checking the first screen 521 by herself so as to place her nipple 202 inside the center circle 524. Thus, the subject 200 adjusts the positions of the breast 201 and the nipple 202 promptly and intuitively on her own.

The first display unit 52 (FIG. 7) indicates an arrow 525 to guide the position of the nipple 202 toward the center circle 524 based on the video image or the still image shot with the camera 25 (FIG. 4). In the example shown in FIG. 8, the position of the nipple 202 (not shown in FIG. 8) is located on the right side of the center circle 524. Accordingly, the first display unit 52 indicates the left-pointing arrow 525. The subject 200 possibly spends extra time for adjusting the position of the nipple 202 with the center circle 524 because she feels nervous during the examination. In such a case, the arrow 525 indicating the direction to move her body to adjust position of the nipple 202 is displayed. In this situation, the subject 200 only needs to move intuitively by moving her body to adjust the position of the nipple 202 in accordance with the arrow 525. Thus, a burden on the subject 200 is reduced.

As described above, the first display unit 52 causes the display device 32 (FIG. 7) to display the mirror reversed video image or still image shot with the camera 25. As a consequence, the subject 200 moves her body to adjust the position of the nipple 202 by the same movement as a movement she would make when she looks at a mirror. In this way, the human subject 200 only needs to move intuitively in accordance with the instruction on the first screen 521 and the burden on the subject 200 is thus reduced.

In another embodiment, the breast screening equipment 100 includes the control device 50 (FIG. 7), which is provided with speakers (not shown) located on the right side and the left side, respectively, and a voice generator (not shown). The speakers emit directional sounds audible only to the subject 200. In addition to the display of the arrow 525 by the first display unit 52 or instead of this display, the voice generator causes the speaker on the side corresponding to the direction to move the nipple 202 to emit a guiding voice. In the example in FIG. 8 of urging the leftward movement, the voice generator causes the speaker on the left side to emit a voice such as “please move to the left”. This configuration allows the subject 200 to adjust the position easily even when the subject 200 is taking off the glasses for the examination and unable to see the first screen 521 well, for example.

In still another embodiment, the breast screening equipment 100 is configured to relax the subject 200 by way of at least one of a sense of smell and a sense of touch. Taking the sense of smell as an example, the breast screening equipment 100 includes a device (not shown) that generates a relaxing smell such as lavender aroma. Taking the sense of touch as an example, the breast screening equipment 100 is provided with, for example, a soft material (not shown) which is located within the reach of a hand of the subject 200 so that the subject 200 relaxes herself by continuously touching the soft material during the screening. Meanwhile, in yet another embodiment, a portion of the support table 1 that comes into contact with the subject 200 may be, for example, subjected to an antibacterial treatment or this portion may be formed from a highly deodorant material, so as to reduce uncomfortable feeling of the subject 200 from a hygienic perspective when she lies down on the breast screening equipment 100. Any of (or both of) an air purifier and a sterilization deodorizer not shown may be installed in the vicinity of the breast screening equipment 100.

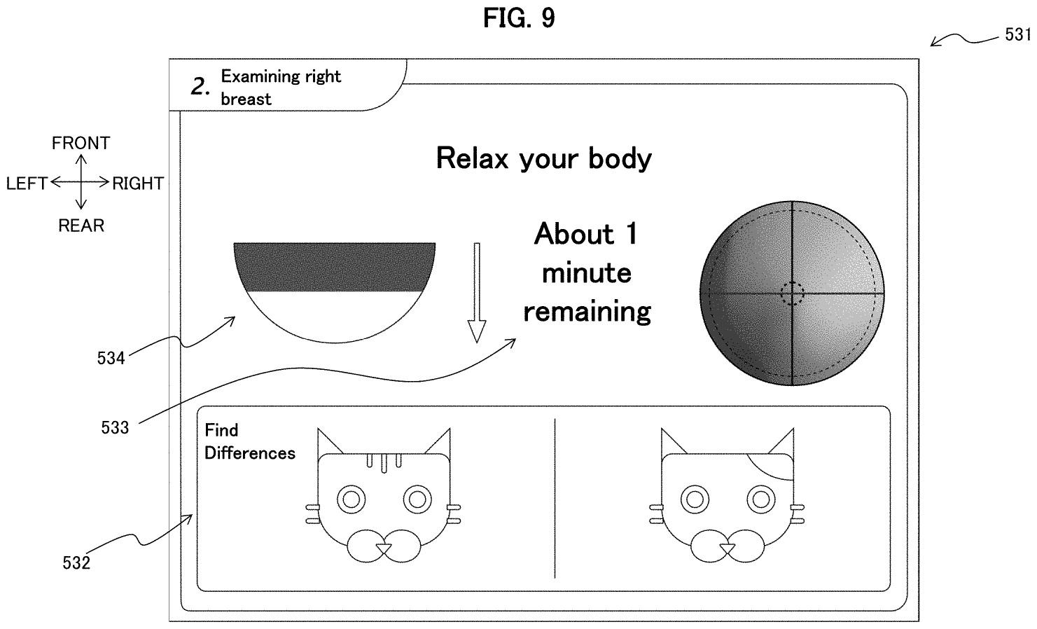

Back to FIG. 7, based on the instruction from the examination unit 51, the second display unit 53 causes the display device 32 to display a second display screen 531 (FIG. 9), which includes at least one of a screen 532 (FIG. 9) to relax the subject 200 during the examination and a screen 533 (FIG. 9) that indicates remaining time of the examination. Provision of the second display unit 53 allows the subject 200 to relieve anxiety that she may feel during the examination, and to recognize the remaining time before completion of the examination, thus reducing the burden on the subject 200 associated with the examination.

FIG. 9 is a schematic diagram of the second display screen 531. In the example shown in FIG. 9, the second display screen 531 includes the screen 532 and the screen 533. The screen 532 includes two pictures located on the right and left and containing slightly different features. The subject 200 may try a “find differences” quiz by using these two pictures during the examination. This makes it possible to relax the subject 200 by letting her concentrate on something other than the examination. In the meantime, it is also possible to prevent the subject 200 from moving the breast 201 by allowing her to concentrate on something other than the examination, and thus to improve accuracy of the examination. The screen 532 may contain other images such as animal videos and landscapes.

The screen 533 indicates the remaining time of the examination located near the center of the second display screen 531. In this way, it is possible to display the remaining time of the examination at the position most recognizable by the subject 200 and thus to reduce the burden on the subject 200. The second display screen 531 further includes a screen 534 that illustrates the progress of the examination. As discussed earlier, the ultrasound examination is carried out by moving the ultrasound transmission reception mechanism 23 (FIG. 2) in the vertical direction. Accordingly, by illustrating a screened region and an unscreened region of the breast 201, the subject 200 intuitively understands the progress of the examination.

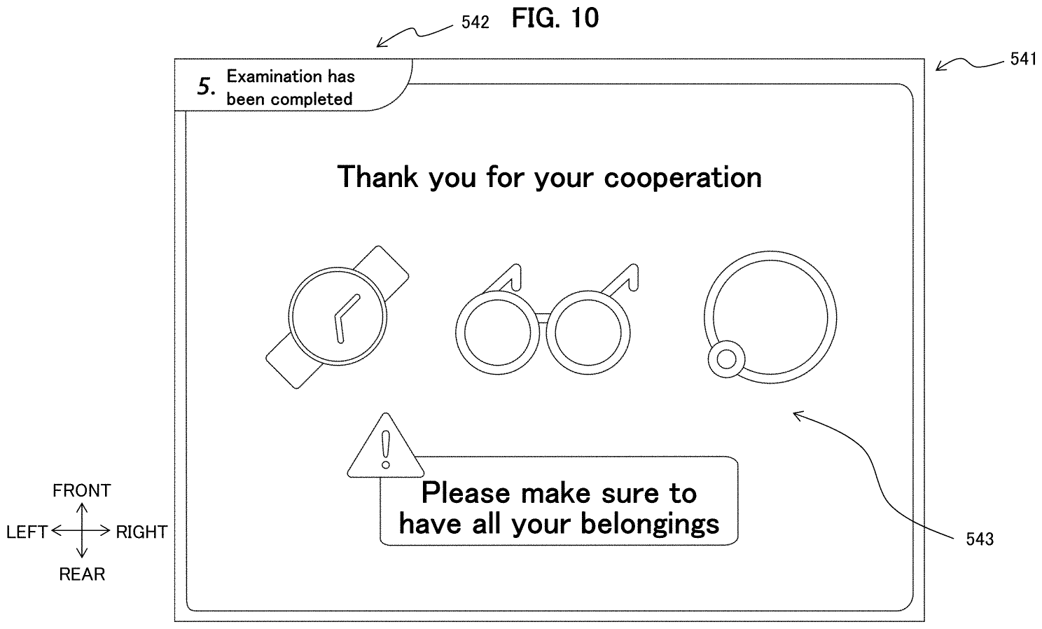

Back to FIG. 7, based on the instruction from the examination unit 51, the third display unit 54 causes the display device 32 to display a third screen 541 (FIG. 10), which informs the subject 200 of completion of the examination when it takes place. Provision of the third display unit 54 enables the subject 200 to recognize the completion of the examination.

FIG. 10 is a schematic diagram of the third screen 541. The third screen 541 includes a sentence 542 to inform of completion of the examination, and moreover, a screen 543 to remind the subject 200 of a watch, glasses, accessories, and the like that she took off prior to the examination and to make sure to put them back on. Thus, it is possible to reduce the chance of these things being left behind.

The above-described breast screening equipment 100 is designed to insert the breast 201 into the cylinder 21 that projects from the upper surface 10. Accordingly, the breast 201 is easily inserted deep into the cylinder 21. As a consequence, it is possible to examine the breast 201 in a wide range in terms of the height direction.

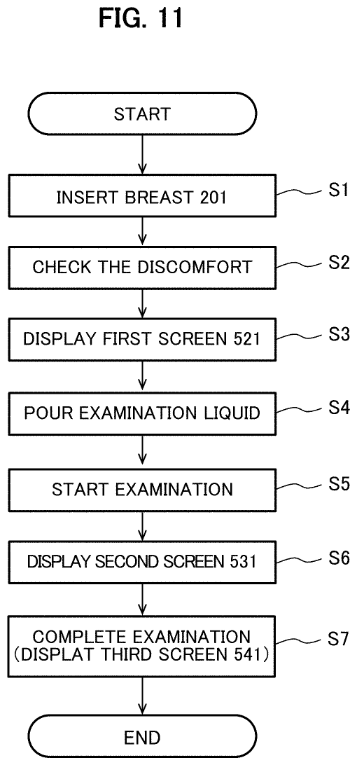

FIG. 11 is a flowchart showing a breast screening method of this embodiment. The flow illustrated in FIG. 11 is executed by the breast screening equipment 100 (FIG. 1).

The subject 200 (FIG. 6) takes a prone position on the cushion 11 (FIG. 1) placed on the upper surface 10 (FIG. 1) and inserts the breast 201 (FIG. 2) into the opening 22 (FIG. 2) (step S1). Thus, the head 203 (FIG. 6) of the subject 200 is located at the position of the window 31 (FIG. 5) to allow the subject 200 to view the display device 32 (FIG. 4). The operator of the breast screening equipment 100 finds out whether or not the subject 200 feels pain (step S2). If the subject 200 feels pain, the operator asks the subject 200 to stand up once so as to replace the cushion 11 with a thicker one, for example. In this way, the operator reduces the height of the upper end surface 213 of the cylinder 21 relative to the upper surface 10.

After the adjustment of the relative height, the subject 200 inserts the breast 201 into the opening 22 again. After the operator finds out that the subject 200 feels no pain or only a bearable pain, the first display unit 52 (FIG. 7) causes the display device 32 to display the first screen 521 (FIG. 8) in accordance with the operation of the input device 70 (FIG. 7) by the operator (step S3). The subject 200 adjusts the positions of the breast 201 and the nipple 202 by herself while confirming the first screen 521. After completion of the position adjustment, the operator operates the input device 70 so as to pour the examination liquid into the cylinder 21 up to the height of the opening 22 (step S4). After the pouring is completed, the examination mechanism 2 (FIG. 7) starts the ultrasound examination of the breast 201 (step S5).

During the examination by the examination mechanism 2, the second display unit 53 (FIG. 7) causes the display device 32 to display the second display screen 531 (FIG. 9) (step S6). Thus, the burden on the subject 200 associated with the examination is reduced by allowing the subject 200 to view the second display screen 531. After completion of the ultrasound transmission and reception covering the entire region of the breast 201 by the examination mechanism 2, the third display unit 54 (FIG. 7) causes the display device 32 to display the third screen 541 (FIG. 10) and the examination is completed (step S7). Accordingly, the subject 200 recognizes the completion of the screening.

What is claimed is:

1. A breast screening equipment comprising:
a support table configured to support a subject in a prone position; and
an examination mechanism comprising a cylinder having an opening at an upper end and protruding from an upper surface of the support table,
wherein
a breast to be examined of the subject is inserted in the opening and
the examination mechanism is configured to conduct an ultrasound examination of the inserted breast in the cylinder.

2. The breast screening equipment according to claim 1, wherein
the cylinder comprises a hollow portion which is located inside of a wall forming the cylinder and extends above the upper surface of the support table, and
the examination mechanism comprises an annular ultrasound transmission reception mechanism being movable inside of the hollow portion vertically.

3. The breast screening equipment according to claim 1, wherein
the cylinder has a height relative to the upper surface of the support table, and
the height is set to a predetermined height that hardly cause pain to the subject.

4. The breast screening equipment according to claim 1, wherein
the height of the cylinder relative to the upper surface of the support table is in a range of from 5 mm or more to 50 mm or below.

5. The breast screening equipment according to claim 1, wherein
the upper surface comprises a first surface configured to support the lower body of the subject, and a second surface on which to put the head of the subject, and
the second surface is located at a lower position than the first surface.

6. The breast screening equipment according to claim 1, wherein
the examination mechanism comprises a flow channel and is configured to conduct the ultrasound examination with an examination liquid in the cylinder, and
the examination liquid overflowing from the opening of the cylinder flows through the flow channel to the outside of the cylinder.

7. The breast screening equipment according to claim 6, wherein
the examination mechanism comprises a receiving portion surrounding at least a part of the cylinder and configured to receive the examination liquid flowing out through the flow channel.

8. The breast screening equipment according to claim 1 further comprising a display device located at a position corresponding to the head of the subject and being visible to the subject.

9. The breast screening equipment according to claim 8 further comprising a control device including a first display unit wherein
the first display unit is configured to display on the display device a first screen that guides a position of at least any one of a breast and a nipple of the subject in the prone position.

10. The breast screening equipment according to claim 8 further comprising a control device including a second display unit wherein
the second display unit configured to display on the display device a second screen that includes at least any one of a screen making the subject relax during the examination and a screen indicating a remaining time of the examination.

11. The breast screening equipment according to claim 2, wherein
the cylinder has a height relative to the upper surface of the support table, and
the height is set to a predetermined height that hardly cause pain to the subject.

12. The breast screening equipment according to claim 2, wherein
the height of the cylinder relative to the upper surface of the support table is in a range of from 5 mm or more to 50 mm or below.

13. The breast screening equipment according to claim 2, wherein
the upper surface comprises a first surface configured to support the lower body of the subject, and a second surface on which to put the head of the subject, and
the second surface is located at a lower position than the first surface.

14. The breast screening equipment according to claim 2, wherein
the examination mechanism comprises a flow channel and is configured to conduct the ultrasound examination with an examination liquid in the cylinder, and
the examination liquid overflowing from the opening of the cylinder flows through the flow channel to the outside of the cylinder.

15. The breast screening equipment according to claim 2 further comprising a display device located at a position corresponding to the head of the subject and being visible to the subject.
